(19) United States
(12) Patent Application Publication
(10) Pub. No.: US 2009/0204126 A1
(43) Pub. Date: Aug. 13, 2009

(54) Cutting Instrument

(76) Inventor: Andrew Le, New South Wales (AU)

Correspondence Address: SCHWEGMAN, LUNDBERG & WOESSNER, P.A., P.O. BOX 2938, MINNEAPOLIS, MN, 55402, US

(21) Appl. No.: 11/992,352

(22) PCT Filed: Sep. 20, 2006
(86) PCT No.: PCT/AU2006/001375

§ 371 Date: Jan. 9, 2009

(30) Foreign Application Priority Data
Sep. 21, 2005 (AU) 2005905212

(51) Int. Cl. A61B 17/10

(52) U.S. Cl. 606/138

(57) ABSTRACT

A cutting instrument (10) comprises a generally elongate handle (12) configured to be gripped by a user; and an elongate blade (14) generally axially aligned with the handle, and having a proximal end connected to the handle and a distal end, the blade having a tip (32) on the distal end thereof, a portion (22) of the blade adjacent the distal end being of generally planar configuration, one edge of said portion defining a bearing edge (30), and the opposite edge of said portion defining a cutting edge (28), the blade having a barb formation (24) rearward of the tip adjacent the cutting edge, said barb formation having a non-cutting edge (36) facing the distal end, and a sharp edge (26) facing the proximal end and defining a continuation of said cutting edge (28).

### FIELD OF THE INVENTION

The present invention relates to a cutting instrument. In particular, although not exclusively, the invention relates to a cutting instrument for removal of surgical stitches or sutures. The invention also relates to a cutting instrument which may be used for cutting subcutaneous fibrous bands such as can be found in rolling acne scars or nasolabial folds and other facial wrinkles.

### BACKGROUND OF THE INVENTION

Sutures used in surgical procedures are generally in the form of a loop of thread inserted through the tissue which is tied with a suture knot. In cosmetic surgery, particularly face or eye surgery, such sutures are particularly small and difficult to see, since by the time of their removal they are often overgrown with skin. The fact that they are difficult to see makes removal of the sutures difficult. A common problem with removal of sutures is that the knife used to cut the sutures cuts both sides of the loop so that the outermost portion of the suture between the two cuts may be removed leaving behind the remaining part of the loop between the two cuts. This can lead to infection, foreign body reaction, granuloma formation and consequential bad scarring.

It is therefore an object of the present invention to provide a cutting instrument which overcomes or at least addresses some of the aforementioned disadvantages.

The foregoing prior art discussion is not to be taken as an admission of common general knowledge.

### SUMMARY OF THE INVENTION

According to the invention there is provided a cutting instrument comprising:

a generally elongate handle configured to be gripped by a user; and

an elongate blade generally axially aligned with the handle, and having a proximal end connected to the handle and a distal end, the blade having a tip on the distal end thereof, a portion of the blade adjacent the distal end being of generally planar configuration, one edge of said portion defining a bearing edge, and the opposite edge of said portion defining a cutting edge, the blade having a barb formation rearward of the tip adjacent the cutting edge, said barb formation having a forward facing edge which is a non-cutting edge, and a rearward facing edge which is sharp and defines a continuation of said cutting edge.

Preferably the bearing edge is of arcuate convex shape. The cutting edge rearward of said barb is preferably of arcuate convex shape.

The pointed tip is preferably of rounded so as to limit the possibility of accidentally piercing the skin in use.

It is preferred that the blade comprises a generally planar portion on which the barb and cutting edges are provided, and a connecting portion, preferably integral with the planar portion, for connection to the handle. The connecting portion may be in the form of a shaft. The shaft may be generally curved along its length. The cutting instrument is intended to be particularly small to deal with small sutures used in cosmetic surgery. In a preferred form of the invention, the blade will fit into an envelope 50 mm long and 5 mm wide. In one arrangement the length of the cutting edge is no longer than 10 mm long. The blade may be less than 3 mm in width at its widest point. While the blade is preferably integral from the proximal to the distal end, it is possible for the distal end of the blade to be formed separately and subsequently joined to a shaft. Preferably, the proximal end of the blade is joined directly to the handle. However, intermediate parts may be provided. The handle may be configured to allow the blade to be replaced.

The handle is preferably cylindrical. In a most preferred form, a recess may be provided at the distal end of the handle to serve as a thumb locator for the user of the cutting instrument. The remainder of the handle may be provided with a gripping surface. The handle may be integrally formed with the blade.

The invention extends to a blade adapted to be used with a cutting instrument as above defined.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

In order that the invention may be more fully understood, one embodiment will now be described with reference to the figures in which:

FIG. 1 is a top view of a handle of the cutting instrument according to a preferred embodiment of the present invention;

FIG. 2 is a side view of the handle of FIG. 1;

FIG. 3 is a top view of the cutting instrument according to the preferred embodiment;

FIG. 4 is a side view of the cutting instrument of FIG. 3;

FIG. 5 is a detailed top view of the distal end of the cutting instrument shown in FIG. 4;

FIG. 6 is a side view of the cutting instrument shown in FIG. 5;

FIG. 7 is a schematic view illustrating the cutting instrument of FIG. 3 in use to remove a suture;

FIGS. 8 to 11 are detailed schematic views showing the distal end of the cutting instrument of FIG. 7 in removal of a suture;

FIG. 12 is a schematic view illustrating the use of the cutting instrument of FIG. 3 to subscise a rolling acne scar;

FIG. 13 is a schematic view of the scar tissue after subscision;

FIG. 14 is a schematic view showing the use of the instrument of FIG. 3 to subscise nasolabial folds and other facial wrinkles;

FIG. 15 is a schematic view showing the fold of FIG. 14 after subscision.

### DESCRIPTION OF THE EMBODIMENT

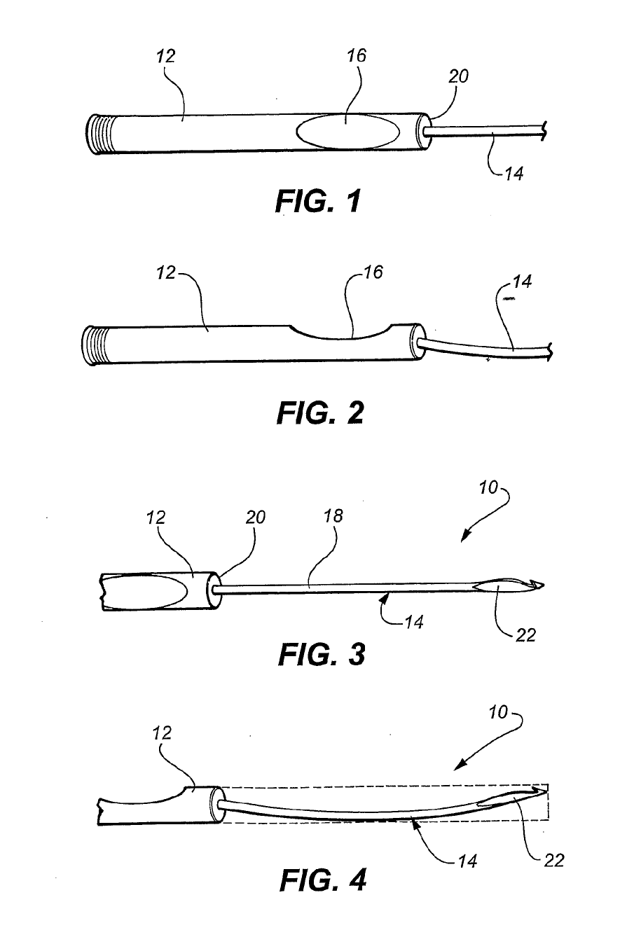

FIGS. 1 to 4 show the basic form of the cutting instrument 10 which includes a handle 12 and a blade 14. The handle 12 may be made of plastic or metal and as shown has a cylindrical shape. Near the distal end 20 of the handle, a concave thumb indention 16 is provided to aid the user in holding the instrument 10. The preferred length of the handle is about 50 mm with a diameter of about 5 mm. The thumb indentation 16 may be formed about 3 mm from the distal end 20 of the handle, extending for about 15 mm.

The blade 14 is suitably made of surgical quality stainless steel and comprises a planar portion 22, and a connecting portion 18 which is in the form of a shaft. The shaft 18 is joined to the handle 12 at the distal end 20 of the handle.

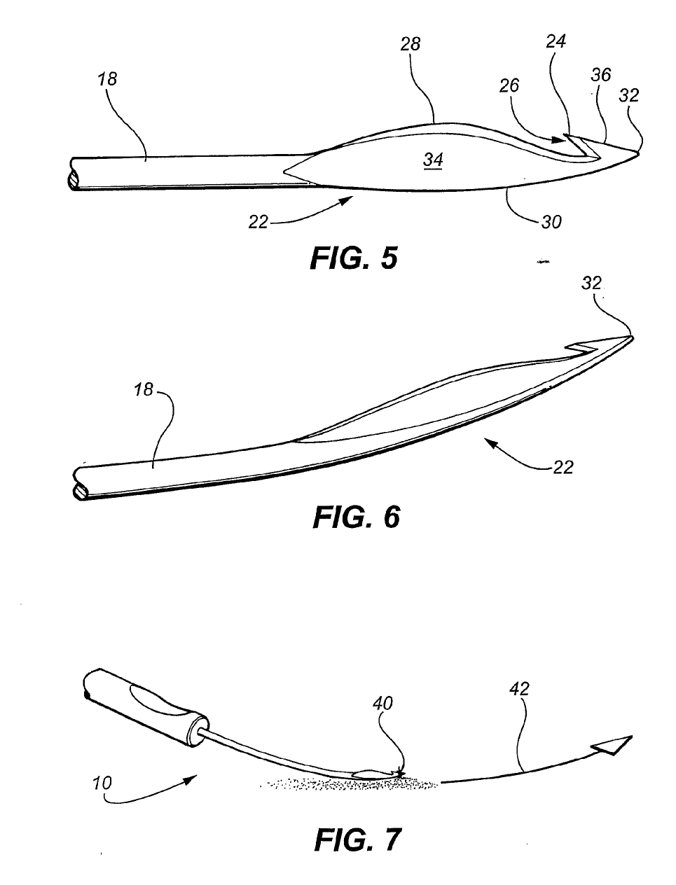

The planar portion 22 of the blade 14 is shown more clearly in FIGS. 5 and 6. The planar portion 22 is provided with a barb 24 which is set back from the distal end of the planar portion 22. The planar portion 22 has a pointed tip 32, and a cutting edge 28 on one longitudinal edge thereof, and a non-cutting bearing edge 30 on the opposite longitudinal edge thereof. It will be noted that both the cutting edge 28 and the bearing edge 30 are of arcuate convex shape, and the edges 28 and 30 converge to meet at the tip 32. It is envisaged that the tip 32 is rounded, with a radius as small as 0.1 mm or even smaller so that the tip can easily slide through the small suture loop with no visible gap. Also the small round tip will enhance skin penetration for sub-cutaneous procedures referred to below with little discomfort to the skin. This is because the rounded non-cutting tip will only split the skin surface to go in without cutting the skin.

The barb 24 is rearward of the tip 32, on the cutting edge side of the planar portion 22. The barb 24 has a forward facing edge 36 which is a non-cutting edge, and a rearward facing or undercut edge 26 which forms a cutting edge, as is described in more detail below. The cutting edge 26, and the cutting edge 28 form a continuous cutting edge rearward of the barb 24.

As can be seen in FIGS. 4 and 6, the bearing edge 30 is curved and defines a bearing surface which is convex in shape. The convex shape of the bearing edge 30 extends from the tip 32 the length of the blade 14, to the point where the blade 14 joins with the handle 12.

Taking into account the curvature of the blade 14, it is intended that the blade will fit into a conceptual envelope which is 50 mm by 5 mm. Further, as can be seen from an inspection of FIGS. 3 and 4, the whole of the line of curvature of the shaft 18 lies in the vertical plane, where the plane of the page in FIG. 4 is regarded as the vertical plane.

As mentioned, the shaft 18 is of circular cross-section however, at the planar portion 22, the blade is flattened out or planar, defining blade faces 34. The planar portion of the blade has a length of about 10 mm and is about 2 mm wide.

The use of the cutting instrument 10 for the removal of sutures, particularly small cosmetic sutures, will now be described with reference to FIGS. 7 to 11. The pointed tip 32 of the instrument 10 is easily able to slide through the smallest gap, i.e. typically 0.1 mm, of suture loop 40. A number of features of the cutting instrument 10 enhance patient safety in use of the instrument. The first safety feature is the horizontally oriented cutting edges 26, 28 which preclude the patient from being accidentally cut when the cutting instrument is applied to the patient's skin surface. Furthermore, the curvature of the blade 14 will assist in avoiding accidental skin puncture by the tip 32. In particular, the curvature of the blade 14 is such as to extend from the proximal end away from the longitudinal axis of the handle 12 and then towards and beyond the longitudinal axis such that the tip 32 is not in line with the longitudinal axis. This allows the distal end of the blade 14 to make contact with the skin with the handle being held above the skin by an operator, in a manner whereby the tip 32 does not point in a direction towards the patient's skin. In FIG. 7, arrow 42 illustrates an arcuate motion of the blade that results when the blade is pushed forward along the skin surface. The upwardly oriented motion 42 helps to avoid accidental puncturing of the skin.

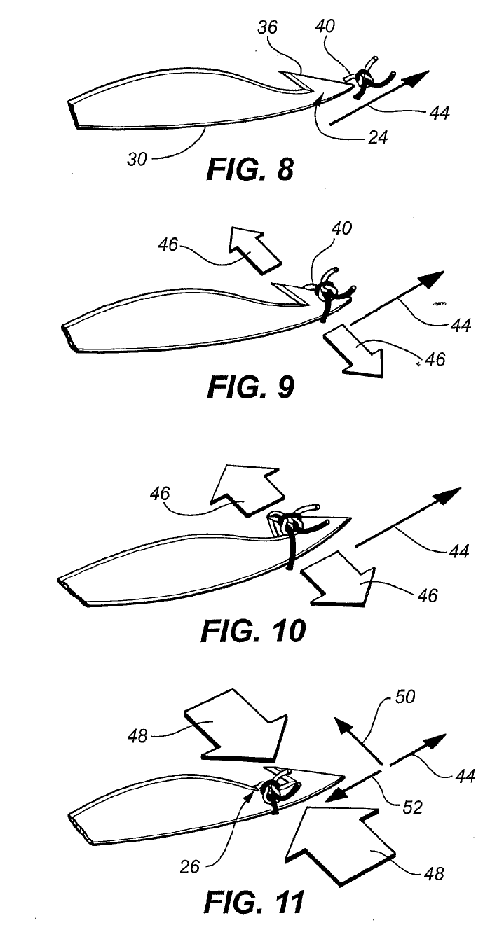

FIG. 8 illustrates the insertion of the tip 32 through the suture loop 40. The tip 32 is moved generally in the direction indicated by arrow 44. Because the tip is bordered by two non-cutting edges 36 and 30, accidental severing of both arms of the loop 40 is avoided. The cutting instrument is only operable to cut the loop when the barb 24 is disposed within the loop. When the cutting instrument is disposed outside the loop, no cutting mechanism will operate since the tip of the blade is free to move in at least three directions without cutting the loop, i.e. sideways, forwards/backwards and up/down. Such movement outside the loop is fairly free and this will provide tactile input to the operator, in addition to visual input that the cutting instrument has not yet properly engaged the suture loop 40.

FIG. 9 shows the tip advancing through the loop 40. In advancing, both arms of the loop are stretched as indicated by arrows 46 and the loop will thus be under tension. Referring to FIG. 10, as the cutting instrument continues to advance in direction 44, the edges 30, 36 will not cut the loop but will stretch the loop 40 to its maximum extent.

As shown in FIG. 11, when the loop 40 passes over the widest part of the barb, the tension within the loop will cause the sides of the loop to snap towards the cutting edge 26, as illustrated by arrows 48. This movement 48 may cut in full or in part, the white arm of the loop 40 as shown. On the other hand, the loop 40 need not necessarily sever at this point. If not, then the barb 24 is trapped inside the loop 40, providing tactile input in addition to the operator's visual input that the barb is correctly in position. If the user moves the cutting instrument in any of the directions indicated by the arrows 44, 50, 52 in FIG. 11 then the loop will be cut, but only in one place. The operator may then withdraw the whole of the severed suture out of the patient.

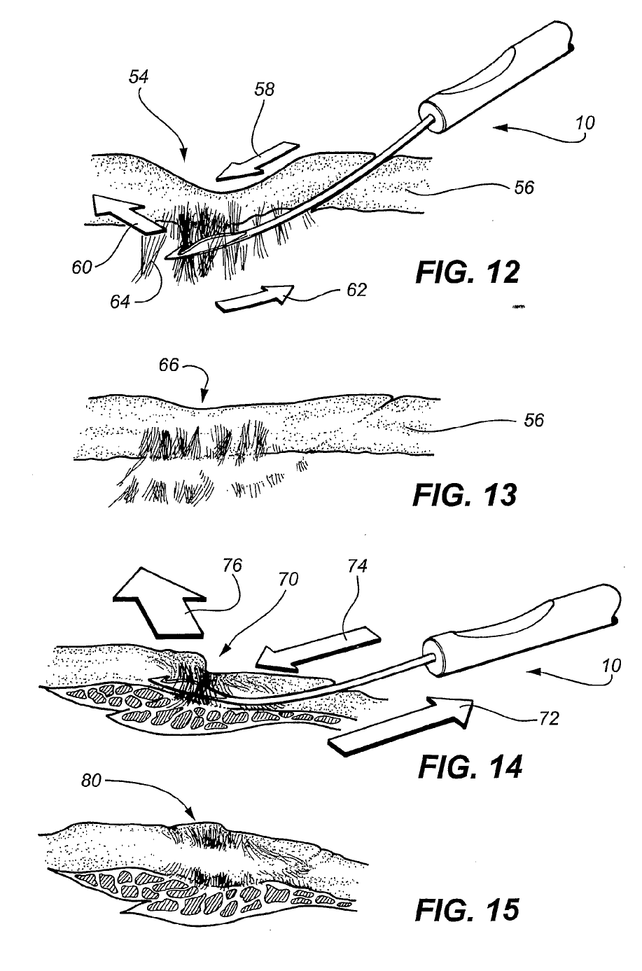

The versatility of the cutting instrument 10 can be seen from FIGS. 12 to 15. The cutting instrument 10 can be used to subscise rolling acne scars and nasolabial folds and other facial wrinkles. In rolling acne scar 54, subcutaneous fibrotic strands 64 deform the skin 56 to form the scar 54. The cutting instrument 10 can be used in three actions to cut the subcutaneous fibrotic strands 64. Firstly, the instrument 10 may be used in a lancing action 58 which means forward cutting similar to thrusting a sword. The instrument 10 may also be used in a fanning motion 60 which is sideways cutting. Furthermore, the cutting instrument can also be used by pulling 62. The pulling action will cause the fibrotic strands 64 to be caught and cut by cutting edge 26 below the barb 24.

FIG. 13 illustrates the beneficial effects of the subscision. The rolling acne scar 66 after subscision is less pronounced than the original scar 54.

In a similar manner, the cutting instrument 10 may be operable on nasolabial folds and other facial wrinkles. FIG. 14 illustrates a procedure on fold 70. As before, the cutting instrument 10 may be used subcutaneously in a lancing motion 74, a fanning motion 76 and a pulling motion 72. FIG. 15 illustrates the resulting state 80 of fold 70 after the subscision.

It will be appreciated that because of the relatively small dimensions of the blade, typically 2 mm wide at its widest point, the blade is able to penetrate below the skin of the patient without causing any significant entry wound. Also the non-cutting edges 30, 36 ensure that minimum inadvertent damage is caused in moving the instrument to the point below the skin surface where subscising is to be undertaken.

The foregoing describes only one embodiment of the present invention and modifications may be made thereto without departing from the scope of the present invention.

1-21. (canceled)

22. A cutting instrument comprising:
a generally elongate handle configured to be gripped by a user; and
an elongate blade generally axially aligned with the handle, and having a proximal end connected to the handle and a distal end, the blade having a tip on the distal end thereof, a portion of the blade adjacent the distal end being of generally planar configuration, one edge of said portion defining a bearing edge, and the opposite edge of said portion defining a cutting edge, the blade having a barb formation rearward of the tip adjacent the cutting edge, said barb formation having a non-cutting edge facing the distal end, and a sharp edge facing the proximal end and defining a continuation of said cutting edge.

23. A cutting instrument as claimed in claim 22 wherein the bearing edge is of arcuate convex shape.

24. A cutting instrument as claimed in claim 22 wherein the cutting edge rearward of said barb formation is of arcuate convex shape.

25. A cutting instrument as claimed in claim 22 wherein the tip is rounded.

26. A cutting instrument as claimed in claim 22 wherein the blade comprises a connecting portion for connection to the handle and extending from the proximal end to the planar portion on which the barb and cutting edges are provided.

27. A cutting instrument as claimed in claim 26 wherein the connecting portion includes a shaft.

28. A cutting instrument as claimed in claim 26 wherein the connecting portion is curved along its length.

29. A cutting instrument as claimed in claim 28 wherein the curvature of the connecting portion lies within a plane.

30. A cutting instrument as claimed in claim 22 wherein the cutting instrument is sufficiently small for insertion of the barb into a small suture used in cosmetic surgery.

31. A cutting instrument as claimed in claim 22 wherein the blade fits into an envelope 50 mm long and 5 mm wide.

32. A cutting instrument as claimed in claim 22 wherein a length of the cutting edge is less than 10 mm.

33. A cutting instrument as claimed in claim 22 wherein the blade is less than 3 mm in width.

34. A cutting instrument as claimed in claim 26 wherein the planar portion of the blade is integrally formed with the connecting portion of the blade.

35. A cutting instrument as claimed in claim 22 wherein the blade is replaceably connected to the handle.

36. A cutting instrument as claimed in claim 22 wherein the handle is cylindrical.

37. A cutting instrument as claimed in claim 22 wherein a recess is provided at a distal end of the handle to serve as a thumb locator for a user of the cutting instrument.

38. A cutting instrument as claimed in claim 37 wherein the handle includes a gripping surface.

39. A cutting instrument as claimed in claim 22 wherein the handle is integrally formed with the blade.

40. An elongate blade for use with a cutting instrument, the blade having a proximal end adapted to be connected to a handle of the cutting instrument and a distal end, the blade having a tip on the distal end thereof, a portion of the blade adjacent the distal end being of generally planar configuration, one edge of said portion defining a bearing edge, and the opposite edge of said portion defining a cutting edge, the blade having a barb formation rearward of the tip adjacent the cutting edge, said barb formation having a non-cutting edge facing the distal end, and a sharp edge facing the proximal end and defining a continuation of said cutting edge.
